(19) United States
(12) Patent Application Publication
(10) Pub. No.: US 2025/0221909 A1
(43) Pub. Date: Jul. 10, 2025

(54) HAND SOAP COMPOSITIONS WITH MAILLARD REACTION INHIBITING ENZYME

(71) Applicant: HENKEL AG & CO. KGAA, Düsseldorf (DE)

(72) Inventors: Daniel Thomas PIORKOWSKI, Fairfield, CT (US); Joseph D. DAHLMEYER, JR., East Haven, CT (US)

(21) Appl. No.: 18/990,105

(22) Filed: Dec. 20, 2024

Related U.S. Application Data
(60) Provisional application No. 63/619,597, filed on Jan. 10, 2024.

(51) Int. Cl. A61K 8/66; A61K 8/41; A61K 8/60; A61Q 19/10

(52) U.S. Cl. CPC A61K 8/66; A61K 8/41; A61K 8/60; A61Q 19/10

(57) ABSTRACT

Hand soap compositions having an increased shelf-life are described herein. The hand soap compositions include a Maillard reaction inhibiting enzyme that prevents a Maillard reaction between amine source and reducing sugars present in a hand soap composition. The Maillard reaction can cause yellowing of the hand soap composition. Hand soap compositions described herein, which include the Maillard reaction inhibiting enzyme, do not undergo yellowing and thus have an increased shelf-life compared to equivalent hand soap compositions that lack such an enzyme.

### CROSS-REFERENCE TO RELATED APPLICATION

This application claims the benefit of U.S. Provisional Patent Application 63/619,597, filed Jan. 10, 2024, the entire contents of which are incorporated by reference herein.

### BACKGROUND OF THE INVENTION

The present disclosure relates to personal care compositions, and more particularly hand soap compositions having an increased shelf-life.

In hand soap compositions, there is an ongoing trend to include sustainable materials in formulating the hand soap. Sustainable materials are derived from renewable, recycled and waste carbon sources and their combination, which at the end of life can be recycled, biodegraded, or composted. Sustainable materials also exhibit reduced environmental impact throughout their life cycle. The use of sustainable materials within hand soap compositions can reduce the cost of formulating hand soap compositions and can provide an environmentally friendly product.

Reducing sugars undergo a Maillard reaction and cause browning in products when in the presence of an amine and heat. Amines can be found in hand soap compositions from a variety of sources including, for example, proteins and surfactants such as quaternary ammonium compounds, cetrimonium chloride, dimethyl myristamine and lauramidopropylamine oxide. Browning reactions cause hand soap compositions to turn yellow over time, especially when stored at elevated temperatures such as, for example, from 35° C. to 50° C. In some cases, it has been observed that as little as 40 ppm of reducing sugars can cause browning reactions to occur within a hand soap composition over time.

The discoloration, i.e., yellowing, of hand soap compositions caused by the Maillard reaction can lead to consumer dissatisfaction and consumer apprehension that the hand soap composition is no longer useable. Thus, there is a need for providing non-yellowing hand soap compositions.

### SUMMARY OF THE INVENTION

Various aspects of the disclosure are directed to hand soap compositions having increased shelf lives. Hand soap compositions according to various aspects of the disclosure include an enzyme that prevents or inhibits a Maillard reaction from occurring in said hand soap compositions. Such an enzyme is referred to herein as a “Maillard reaction inhibiting enzyme”. According to various aspects of the present disclosure, a Maillard reaction is a chemical reaction between an amine and a reducing sugar, which can create melanoidins. Melanoidins are brown, high molecular weight heterogeneous polymers that are formed when reducing sugars and amines combine through the Maillard reaction. The formation of such polymers can cause browning to occur within a hand soap composition. Hand soap compositions according to the disclosure which include a Maillard reaction inhibiting enzyme do not undergo yellowing and thus exhibit increased shelf-lives compared to equivalent hand soap compositions that lack such an enzyme. Due to the prevention of yellowing that would regularly occur prior to the use of said composition by a consumer, consumer confidence in hand soap compositions according to the disclosure will be greater than for equivalent hand soap compositions that lack a Maillard reaction inhibiting enzyme.

In some instances, hand soap compositions according to various aspects of the disclosure comprise, consist essentially of, or consist of, at least one surfactant; an amine source; a sustainable raw material including a reducing sugar; a Maillard reaction inhibiting enzyme that inhibits reaction between the amine source and the reducing sugar; a primary humectant; and at least 40 weight percent water, based on the total weight of the hand soap compositions.

In some instances, the at least one surfactant of hand soap compositions according to the disclosure comprises a primary surfactant. In some instances, the primary surfactant is a key foaming/cleaning agent in the hand soap composition. In some instances, the primary surfactant comprises, consists essentially of, or consists of at least one anionic surfactant. In some instances, the primary surfactant is present in an amount from about 1 weight percent to about 40 weight percent, based on the total weight of the hand soap composition.

In some instances, the at least one surfactant of hand soap compositions according to the disclosure comprises a primary surfactant and a secondary surfactant. In some instances, the secondary surfactant is a surfactant that is used to enhance the foaming and cleaning abilities of the primary surfactant, while also adding a layer of mildness to the hand soap composition and even reducing potential skin irritation. In some instances, the secondary surfactant comprises, consists essentially of, or consists of an amphoteric surfactant, a zwitterionic surfactant, or any combination thereof. In some instances, the secondary surfactant is present in an amount from about 0.1 weight percent to about 15 weight percent, based on the total weight of the hand soap composition.

In some instances, the amine source of hand soap compositions according to the disclosure comprises a protein, an amine containing surfactant, a chelating agent, an amine containing enzyme, or hydrolyzed protein. In some instances, the amine source of hand soap compositions according to disclosure comprises a combination of at least two of a protein, an amine containing surfactant, a chelating, an amine containing enzyme, or a hydrolyzed protein.

In some instances, the amine source of hand soap compositions according to the disclosure is present in an amount from about 0.05 weight percent to about 30 weight percent, based on the total weight of the hand soap composition.

In some instances, the sustainable raw material of hand soap compositions according to the disclosure comprises a sustainable polymer, a sustainable solvent, a secondary humectant, or any combination thereof.

In some instances, the sustainable raw material of hand soap compositions according to disclosure is a sustainable solvent, where the sustainable solvent comprises, consists essentially of, or consists of, sorbitol, xylitol, mannitol, lactitol, isomalt, maltitol, a hydrogenated starch hydrolysate, sucrose, stachyose, verbascose, trehalose, raffinose, or any combination thereof.

In some instances, the sustainable raw material of hand soap compositions according to the disclosure is a sustainable polymer, where the sustainable polymer comprises, consists essentially of, or consists of, a starch, a glycogen, a galactogen, a cellulose, a chitin, or any combination thereof.

In some instances, the sustainable raw material of hand soap compositions according to disclosure, is a secondary humectant, where the secondary humectant comprises, consists essentially of, or consists of an alkyl glucose ether, such as, for example, methyl glycose ether.

In some instances, the Maillard rection inhibiting enzyme comprises hexose oxidase, fructosamine oxidase, fructosamine kinase, carbohydrate oxidase or combinations thereof.

In some instances, the Maillard reaction inhibiting enzyme is present in an amount of from about 0.001 weight percent to about 3 weight percent, based on the total weight of the hand soap composition.

In some instances, hand soap compositions according to various aspects of the disclosure comprises, consists essentially of, or consists of, at least one surfactant; a primary amine source comprising a fermented raw material that includes a reducing sugar; a Maillard reaction inhibiting enzyme that inhibits reaction between the primary amine source and the reducing sugar; a primary humectant; and at least 40 weight percent water, based on the total weight of the hand soap composition.

In some instances where hand soap compositions according to the disclosure include a primary amine source, the at least one surfactant may comprise a primary surfactant. In some instances, the primary surfactant is present in an amount from about 1 weight percent to about 40 weight percent, based on the total weight of the hand soap composition.

In some instances where hand soap compositions according to the disclosure include a primary amine source, the at least one surfactant may comprise a primary surfactant and a secondary surfactant. In some instances, the secondary surfactant is present in an amount from about 0.1 weight percent to about 15 weight percent, based on the total weight of the hand soap composition.

In some instances where hand soap compositions according to the disclosure include a primary amine source, the primary amine source comprises a biosurfactant such as, for example, sophorolipid or rhamnolipid.

In some instances, the primary amine source is present in an amount from about 0.05 weight percent to about 30 weight percent, based on the total weight of the hand soap composition.

In some instances where hand soap compositions according to the disclosure include a primary amine course, the Maillard rection inhibiting enzyme may comprise, consist essentially of, or consist of fructosamine oxidase, fructosamine kinase, carbohydrate oxidase, or any combination thereof.

In some instances, where hand soap compositions according to the disclosure include a primary amine source, the Maillard inhibiting enzyme is present in an amount of from about 0.001 weight percent to about 3 weight percent, based on the total weight of the hand soap composition.

In some instances, where hand soap compositions according to the disclosure include a primary amine source, the hand soap composition further comprises a sustainable raw material including a reducing sugar. In some instances, the sustainable raw material including the reducing sugar comprises a sustainable polymer, a sustainable solvent, a secondary humectant, or any combination thereof. In some instances, the sustainable polymer comprises a starch, a glycogen, a galactogen, a cellulose, a chitin, or any combination thereof. In some instances, the sustainable solvent comprises sorbitol, xylitol, mannitol, lactitol, isomalt, maltitol, a hydrogenated starch hydrolysate, sucrose, stachyose, verbascose, trehalose, raffinose, or any combination thereof. In some instances, the secondary humectant is an alkyl glycose ether such as, for example, methyl glucose ether.

In some instances, the sustainable material (i.e., raw material) used in formulating hand soap compositions according to the disclosure comprises, consists essentially of, or consists of a reducing sugar, such as, for example, fructose or glucose. In some instances, the reducing sugar can be a contaminant (i.e., residual) in the sustainable material or it can be part (i.e., a moiety) of the sustainable material, for example, a monomer of a polymer, which degrades from the sustainable material over time.

Various non-limiting inventive aspects according to the disclosure are as follows:
In some instances, a first aspect of the disclosure can be described as a hand soap composition comprises at least one surfactant, an amine source, a sustainable raw material including a reducing sugar, a Maillard reaction inhibiting enzyme (that inhibits reaction between the amine source and the reducing sugar), a primary humectant, and at least 40 percent water, based on the total weight of the hand soap composition.
In some instances, a second aspect of the disclosure can be described as a hand soap composition according to the first aspect, wherein the at least one surfactant comprises a primary surfactant.
In some instances, a third aspect of the disclosure can be described as a hand soap composition according to the second aspect, wherein the primary surfactant is present in an amount ranging from about 1 weight percent to about 40 weight percent, based on the total weight of the hand soap composition.
In some instances, a fourth aspect of the disclosure can be described as a hand soap composition according to either of the second and third aspects, wherein the at least one surfactant further comprises a secondary surfactant.
In some instances, a fifth aspect of the disclosure can be described as a hand soap composition according to the fourth aspect, wherein the secondary surfactant is present in an amount ranging from about 0.1 weight percent to about 15 weight percent, based on the total weight of the hand soap composition.
In some instances, a sixth aspect of the disclosure can be described as a hand soap composition according to any one of the first through the fifth aspects, wherein the amine source comprises a protein, an amine containing surfactant, a chelating agent, an amine containing enzyme, or a hydrolyzed protein, or any combination thereof.
In some instances, a seventh aspect of the disclosure can be described as a hand soap composition according to any one of the first through the sixth aspects, wherein the amine source is present in an amount ranging from about 0.05 weight percent to about 30 weight percent, based on the total weight of the hand soap composition.
In some instances, an eighth aspect of the disclosure can be described as a hand soap composition according to any one of the first through the seventh aspects, wherein the sustainable raw material comprises a sustainable polymer, a sustainable solvent, or a secondary humectant, or any combination thereof.
In some instances, a ninth aspect of the disclosure can be described as a hand soap composition according to the eighth aspect, wherein the sustainable solvent comprises sorbitol, xylitol, mannitol, lactitol, isomalt, maltitol, a hydrogenated starch hydrolysate, sucrose, stachyose, verbascose, trehalose, or raffinose, or any combination thereof.
In some instances, a tenth aspect of the disclosure can be described as a hand soap composition according to the eighth or ninth aspect, wherein the sustainable polymer comprises a starch, a glycogen, a galactogen, a cellulose, or a chitin, or any combination thereof.
In some instances, an eleventh aspect of the disclosure can be described as a hand soap composition according to any one of the first through the tenth aspects, wherein the Maillard rection inhibiting enzyme comprises fructosamine oxidase, fructosamine kinase, or carbohydrate oxidase, or any combination thereof.
In some instances, a twelfth aspect of the disclosure can be described as a hand soap composition according to any one of the first through the eleventh aspects, wherein the Maillard reaction inhibiting enzyme is present in an amount of from about 0.001 weight percent to about 3 weight percent, based on the total weight of the hand soap composition.
In some instances, a thirteenth aspect of the disclosure can be described as a hand soap composition comprising at least one surfactant, a primary amine source comprising a fermented raw material, where the fermented raw material includes a reducing sugar, a Maillard reaction inhibiting enzyme (that inhibits reaction between the primary amine source and the reducing sugar), a primary humectant, and at least 40 weight percent water, based on the total weight of the composition.
In some instances, a fourteenth aspect of the disclosure can be described as a hand soap composition according to the thirteenth aspect, wherein the at least one surfactant comprises a primary surfactant.
In some instances, a fifteenth aspect of the disclosure can be described as a hand soap composition according to the fourteenth aspect, wherein the primary surfactant is present in an amount ranging from about 1 weight percent to about 40 weight percent, based on the total weight of the hand soap compositions.
In some instances, a sixteenth aspect of the disclosure can be described as a hand soap composition according to either of the fourteenth and fifteenth aspects, wherein the at least one surfactant further comprises a secondary surfactant.
In some instances, a seventeenth aspect of the disclosure can be described as a hand soap composition according to the sixteenth aspect, wherein the secondary surfactant is present in an amount ranging from about 0.1 weight percent to about 15 weight percent, based on the total weight of the hand soap composition.
In some instances, an eighteenth aspect of the disclosure can be described as a hand soap composition according to any one of the thirteenth to the seventeenth aspects, wherein the primary amine source comprises a biosurfactant, an alkyl glucose ether, or a combination of a biosurfactant and an alkyl glucose ether.

In some instances, a nineteenth aspect of the disclosure can be described as a hand soap composition according to any one of the thirteenth to the eighteenth aspects, wherein the primary amine source is present in an amount from about 0.05 weight percent to about 30 weight percent, based on the total weight of the hand soap composition.

In some instances, a twentieth aspect of the disclosure can be described as a hand soap composition according to any one of the thirteenth to the nineteenth aspects, wherein the Maillard rection inhibiting enzyme comprises fructosamine oxidase, fructosamine kinase, or carbohydrate oxidase, or any combination thereof.
In some instances, a twenty-first aspect of the disclosure can be described as a hand soap composition according to any one of the thirteenth to the twentieth aspects, wherein the Maillard inhibiting enzyme is present in the composition in an amount of from about 0.001 weight percent to about 3 weight percent, based on the total weight of the hand soap composition.
In some instances, a twenty-second aspect of the disclosure can be described as a hand soap composition according to any one of the thirteenth to the twenty-first aspects, wherein the hand soap composition further comprises a sustainable solvent including a reducing sugar, a sustainable polymer including a reducing sugar, or a secondary humectant including a reducing sugar, or any combination thereof.
In some instances, a twenty-third aspect of the disclosure can be described as a hand soap composition according to the twenty-second aspect, wherein the sustainable solvent comprises sorbitol, xylitol, mannitol, lactitol, isomalt, maltitol, a hydrogenated starch hydrolysate, sucrose, stachyose, verbascose, trehalose, or raffinose, or any combination thereof.
In some instances, a twenty-fourth aspect of the disclosure can be described as a hand soap composition according to the twenty-second or twenty-third aspect, wherein the sustainable polymer comprises a starch, a glycogen, a galactogen, a cellulose, or a chitin, or any combination thereof.

### BRIEF DESCRIPTION OF THE DRAWINGS

FIG. 1 is an image depicting an exemplary pantone scale for characterization as to the degree of yellowing of detergent compositions comprising reducing sugar agents.

### DETAILED DESCRIPTION OF THE INVENTION

The present disclosure will now be described in greater detail by referring to the following discussion and drawings that accompany the present disclosure. In the following description, numerous specific details are set forth, such as particular structures, components, materials, dimensions, processing steps and techniques, in order to provide an understanding of the various embodiments of the present disclosure. However, it will be appreciated by one of ordinary skill in the art that the various embodiments of the present disclosure may be practiced without these specific details. As used throughout the present disclosure, the term “about” generally indicates no more than ±10%, ═5%, ±2%, ±1% or ±0.5% from a number. When a range is expressed in the present disclosure as being from one number to another number (e.g., 20 to 40), the present disclose contemplates any numerical value that is within the range (e.g., 22, 24, 26, 28.5, 31, 33.5, 35, 37.7, 39 or 40) or any in amount that is bounded by any of the two values that can be present within the range (e.g., 28.5-35).

The terminology used herein is for the purpose of describing particular embodiments only and is not intended to be limiting of the present disclosure. As used herein, the singular forms “a”, “an” and “the” are intended to include the plural forms as well, unless the context clearly indicates otherwise. It will be further understood that the terms “comprises” and/or “comprising”, when used in this disclosure, specify the presence of stated features, integers, steps, operations, elements, and/or components, but do not preclude the presence or addition of one or more other features, integers, steps, operations, elements, components, and/or groups thereof.

According to various aspects of the disclosure, hand soap compositions are provided that have increased shelf-life. Hand soap compositions according to the disclosure comprise a Maillard reaction inhibiting enzyme that inhibits reaction between an amine and a reducing sugar. The presence of such an enzyme inhibits the Maillard reaction between the reducing sugar and the amine and thus the hand soap compositions of the present disclosure have an increased shelf-life. The increased shelf-life is evidenced by the absence of yellowing of the hand soap compositions over an extended period of time. In some instances, the enzyme inhibits the reaction of the reducing sugar with the amine throughout the entire shelf-life of the hand soap composition including at the beginning of the formulation process. These and other aspects of the present disclosure will now be described in greater detail.

According to various aspects of the disclosure, hand soap compositions comprise at least one surfactant. In some instances, the at least one surfactant comprises an anionic surfactant, a nonionic surfactant, a cationic surfactant, an amphoteric surfactant, a zwitterionic surfactant or any combination such surfactants. In some instances, the at least one surfactant comprises a primary surfactant. In some instances, the at least one surfactant comprises a secondary surfactant. In some instances, the primary surfactant is a key foaming/cleaning agent in the hand soap composition. In some instances, the primary surfactant preferably comprises at least one anionic surfactant. In some instances, the secondary surfactant is a surfactant that is used to enhance the foaming and cleaning abilities of the primary surfactant, while also adding a layer of mildness to the hand soap composition and even reducing potential skin irritation. In some instances, the secondary surfactant comprises an amphoteric surfactant, a zwitterionic surfactant, or any combination thereof.

In some instances, the surfactant (primary or secondary) that can be included in the hand soap compositions comprise any surfactant that is dermatologically compatible with skin. By “dermatologically compatible” it is meant that the compound does not cause irritation when applied to the skin and/or hair for the purpose of cleaning and then is subsequently removed within about 30 minutes of application.

In some instances, the primary surfactant is preferably present at a level of from about 1 weight percent to about 40 weight percent, more preferably from about 3 weight percent to about 30 weight percent, and most preferably from about 5 weight percent to about 20 weight percent, based on the total weight of the hand soap composition. In some instances, the secondary surfactant is preferably present at a level of from about 0.1 weight percent to about 15 weight percent, more preferably from about 0.2 weight percent to about 10 weight percent, and most preferably from about 0.5 weight percent to about 5 weight percent based on the total weight of the hand soap composition

According to various aspects of the present disclosure, anionic surfactants useful in soap compositions include, for example, alkyl sulfates, alkyl ether sulfates, alkyl ether carboxylates, acyl isethionates, acyl sarcosinates, acyl taurines, or alkali metal, ammonium, or alkanolammonium salts thereof, or combinations thereof. In some instances, the alkyl or acyl groups of such anionic surfactants comprise from about 10 to about 20 carbon atoms per alkyl or acyl group, in some instances, more preferably, from about 12 to about 18 carbon atoms per alkyl or acyl group. In some instances, the alkyl or acyl groups may be linear, branched, saturated or unsaturated or combinations thereof. In some instances, more preferably, the alkyl or acyl groups are linear. A further description of anionic surfactants that can be use in the hand soap composition is provided herein below.

According to various aspects of the disclosure, the anionic surfactant comprises at least one alkyl sulfate, alkyl ether sulfate, salts thereof, or combinations thereof. These surfactants have respectively the general formula (I) and/or the general formula (II):

R₉OSO₃⁻M⁺ (I)

R₉O(C₂H₄O)ₓSO₃⁻M⁺ (II)

where R₉ is an alkyl group of about 10 to about 20 carbon atoms, x is an integer ranging from 1 to about 10, and M⁺ is a water-soluble cation such as an ammonium ion, an alkali metal ion such as sodium or potassium, or an alkylammonium or alkanolammonium ion such as triethylammonium or triethanolammonium. In some instances, R₉ is preferably an alkyl group of about 12 to about 18 carbon atoms. In some instances, x is preferably an integer ranging from 1 to about 4. In some instances, R₉ is preferably an alkyl group of about 12 to about 18 carbon atoms and x is preferably an integer ranging from 1 to about 4.

In some instances, specific examples of preferred anionic surfactants of general formula (I) and/or (II) include, but are limited to, ammonium lauryl sulfate, sodium lauryl sulfate, ammonium laureth sulfate (which may preferably have 1 to about 3 moles ethylene oxide), sodium laureth sulfate (which may preferably have 1 to about 3 moles ethylene oxide), or combinations thereof.

According to various aspects of the disclosure, other suitable anionic surfactants that can be employed in hand soap compositions according to the disclosure are the water-soluble salts of organic, sulfuric acid reaction products conforming to the formula [R¹—SO₃-M] where R¹ is a straight or branched chain, saturated, aliphatic hydrocarbon radical having from about 8 to about 24, preferably from about 10 to about 18, carbon atoms; and M is a cation described hereinbefore. Non-limiting examples of such surfactants are the salts of an organic sulfuric acid reaction product of a hydrocarbon of the methane series, including iso-, neo-, and n-paraffins, having from about 8 to about 24 carbon atoms, preferably about 12 to about 18 carbon atoms and a sulfonating agent, e.g., SO₃, H₂SO₄, obtained according to known sulfonation methods, including bleaching and hydrolysis. In some instances, preferred anionic surfactants are alkali metal and ammonium sulfonated C₁₀ to C₁₈ n-paraffins.

According to various aspects of the disclosure, other suitable anionic surfactants that can be employed in hand soap compositions are the reaction products of fatty acids esterified with isethionic acid and neutralized with sodium hydroxide where, for example, the fatty acids are derived from coconut oil or palm kernel oil; sodium or potassium salts of fatty acid amides of methyl tauride in which the fatty acids, for example, are derived from coconut oil or palm kernel oil. Other similar anionic surfactants are described in U.S. Pat. Nos. 2,486,921; 2,486,922; and 2,396,278, the entire contents of each of which are incorporated herein by reference.

In some instances, the anionic surfactants are succinates, examples of which include disodium N-octadecylsulfosuccinate; disodium lauryl sulfosuccinate; diammonium lauryl sulfosuccinate; tetrasodium N-(1,2-dicarboxyethyl)-N-octadecylsulfosuccinate; diamyl ester of sodium sulfosuccinic acid; dihexyl ester of sodium sulfosuccinic acid; and dioctyl esters of sodium sulfosuccinic acid.

According to various aspects of the present disclosure, suitable anionic surfactants that can be employed in hand soap compositions include olefin sulfonates having about 10 to about 24 carbon atoms. In this context, the term “olefin sulfonates” refers to compounds which can be produced by the sulfonation of alpha-olefins by means of uncomplexed sulfur trioxide, followed by neutralization of the acid reaction mixture in conditions such that any sulfonates which have been formed in the reaction are hydrolyzed to give the corresponding hydroxy-alkanesulfonates. In some instances, the sulfur trioxide can be liquid or gaseous, and is usually, but not necessarily, diluted by inert diluents, for example by liquid SO₂, chlorinated hydrocarbons, etc., when used in the liquid form, or by air, nitrogen, gaseous SO₂, etc., when used in the gaseous form. In some instances, the alpha-olefins from which the olefin sulfonates are derived are mono-olefins having from about 10 to about 24 carbon atoms, preferably from about 12 to about 16 carbon atoms. In some instances, the alpha-olefins are straight chain olefins. In some instances, in addition to the true alkene sulfonates and a proportion of hydroxy-alkanesulfonates, the olefin sulfonates can contain minor amounts of other materials, such as alkene disulfonates depending upon the reaction conditions, proportion of reactants, the nature of the starting olefins and impurities in the olefin stock and side reactions during the sulfonation process. A non-limiting example of such an alpha-olefin sulfonate mixture is described in U.S. Pat. No. 3,332,880, the entire contents of which are incorporated herein by reference.

Another class of anionic surfactants that can be employed in hand soap compositions according to the disclosure are the beta-alkyloxy alkane sulfonates. These surfactants conform to the formula (III):

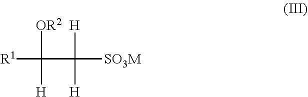

where R¹ is a straight chain alkyl group having from about 6 to about 20 carbon atoms, R² is a lower alkyl group having from about 1 to about 3 carbon atoms, preferably 1 carbon atom, and M is a water-soluble cation as described hereinbefore. In some instances, preferred anionic surfactants for use in the personal care compositions include sodium tridecyl benzene sulfonate, sodium dodecyl benzene sulfonate.

In some instances, the sulfates according to formula (I), the sulfates according to formula (II), the sulfonates of formula (III), and any combinations of these anionic surfactants are preferred anionic surfactants that can be used in the present disclosure as the primary surfactant. In some instances, in which the hand soap composition is mild and sulfate free, the primary surfactant includes one of the secondary surfactants mentioned below as the primary surfactant.

In some instances, hand soap compositions according to the comprise (in addition to an anionic surfactant) a secondary surfactant that includes an amphoteric surfactant, zwitterionic surfactant, or combinations. In some instances, the total amount of amphoteric and zwitterionic surfactant is from 0 to about 5 weight percent and more preferably from about 1 to about 4 weight percent, based on the total weight of the hand soap composition. In some instances, examples of suitable amphoteric or zwitterionic surfactants include, but are not limited to, alkyl betaines, alkylamidobetaines, aminopropionates, aminoglycinates, imidazolinium betaines, sulfobetaines or combinations thereof.

In some instances, preferred zwitterionic surfactants include compounds having formula (IV):

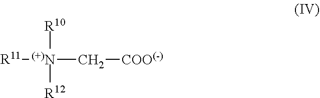

where R¹¹ is an alkyl or alkenyl group containing from about 10 to about 20 carbon atoms or a R¹³—CONH—(CH₂)ₚ group, where R¹³ is an alkyl or alkenyl group containing from about 10 to about 20 carbon atoms and p is an integer ranging from about 2 to about 5, and R¹⁰ and R¹² are alkyl groups containing from 1 to about 5 carbon atoms or hydroxyalkyl groups containing from about 2 to about 4 carbon atoms.

In some instances, preferred amphoteric surfactants include compounds according to formula (V):

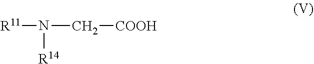

where R¹¹ is an alkyl or alkenyl group containing from about 10 to about 20 carbon atoms or a R₁₃—CONH—(CH₂)ₚ group, where R¹³ is an alkyl or alkenyl group containing from about 10 to about 20 carbon atoms and p is an integer ranging from about 2 to about 5, and R¹⁴ is an alkyl group containing from 1 to about 5 carbon atoms or a hydroxyalkyl group containing from about 2 to about 4 carbon atoms.

In some instances, non-limiting examples of amphoteric or zwitterionic surfactants include sodium 3-dodecyl-aminopropionate, sodium 3-dodecylaminopropane sulfonate, sodium lauroamphoacetate, coco dimethyl carboxymethyl betaine, cocoamidopropyl betaine, cocobetaine, lauryl amidopropyl betaine, oleyl betaine, lauryl dimethyl carboxymethyl betaine, lauryl dimethyl alpha-carboxyethyl betaine, cetyl dimethyl carboxymethyl betaine, lauryl bis-(2-hydroxyethyl) carboxymethyl betaine, stearyl bis-(2-hydroxypropyl) carboxymethyl betaine, oleyl dimethyl gamma-carboxypropyl betaine, lauryl bis-(2-hydroxypropyl)alpha-carboxyethyl betaine, oleamidopropyl betaine, coco dimethyl sulfopropyl betaine, stearyl dimethyl sulfopropyl betaine, lauryl dimethyl sulfoethyl betaine, lauryl bis-(2-hydroxyethyl)sulfopropyl betaine or combinations thereof. In some instances, the zwitterionic or amphoteric surfactant is cocoamidopropyl betaine, oleamidopropyl betaine, or a combination thereof.

According to various aspects of the disclosure, in addition to the above-described components, other common cosmetic additives may be included in hand soap composition according to the disclosure, as long as the basic properties of the hand soap composition are not adversely affected. In some instances, such optional cosmetic additives may be found, for example, in the International Cosmetic Ingredients Dictionary, 7th Edition, 1997, published by the CTFA in Washington D.C, the entire contents of which are incorporated by reference herein. In some instances, the optional components, in total, are present in an amount of from 0 to 20 weight percent, and more preferably from about 0.1 to about 12 weight percent, based on the total weight of the composition.

In some instances, examples of optional additive components include, but are not limited to, one or more surfactants such as nonionic surfactants or cationic surfactants, protein hydrolyzates, other conditioning agents, inorganic electrolyte salts or combinations thereof.

According to various aspects of the present disclosure, one type of nonionic surfactant that can be employed in hand soap compositions of according to the disclosure is an amide. Amides, including alkanol amides, are the condensation products of fatty acids with primary and secondary amines or alkanolamines to yield products of the general formula (VI):

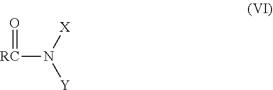

wherein RCO is a fatty acid radical and R is C₈₋₂₀; X is an alkyl, aromatic or alkanol (CHR′CH₂OH wherein R′is H or C₁₋₆ alkyl); Y is H, alkyl, alkanol or X. In some instances, suitable amides include, but are not limited to, cocamide, lauramide, oleamide and stearamide. In some instances, suitable alkanolamides include, but are not limited to, cocamide DEA, cocamide MEA, cocamide MIPA, isostearamide DEA, isostearamide MEA, isostearamide MIPA, lanolinamide DEA, lauramide DEA, lauramide MEA, lauramide MIPA, linoleamide DEA, linoleamide MEA, linoleamide MIPA, myristamide DEA, myristamide MEA, myristamide MIPA, Oleamide DEA, Oleamide MEA, Oleamide MIPA, palmamide DEA, palmamide MEA, palmamide MIPA, palmitamide DEA, palmitamide MEA, palm kernelamide DEA, palm kernelamide MEA, palm kernelamide MIPA, peanutamide MEA, peanutamide MIPA, soyamide DEA, stearamide DEA, stearamide MEA, stearamide MIPA, tallamide DEA, tallowamide DEA, tallowamide MEA, undecylenamide DEA, undecylenamide MEA and PPG-2 hydrodroxyethyl coco/isostearyamide. In some instances, the condensation reaction may be carried out with free fatty acids or with all types of esters of the fatty acids, such as fats and oils, and particularly methyl esters. In some instances, the reaction conditions and the raw material sources determine the blend of materials in the end product and the nature of any impurities.

In some instances, other types of surfactants, not previously described herein, that are typically used in compositions may optionally be added to hand soap compositions according to the disclosure such as nonionic and/or cationic surfactants. In some instances, these surfactants are preferably present in an amount of no more than about 10 weight percent, more preferably in an amount of 0 weight percent to about 5 weight percent, and most preferably in an amount of about 0.01 weight percent to about 3 weight percent, based on the total weight of the composition. In some instances, surfactants may include for example, nonionic surfactants present in an amount preferably ranging from 0 weight percent to about 10 weight percent and more preferably from 0 weight percent to about 2 weight percent, based on the total weight of the composition. In some instances, non-limiting examples of nonionic surfactants are alkyl polyglycosides, alkyl oligoglycosides, ethoxylated fatty alcohols, sorbitan esters, or alkanolamides such as cocamide MEA, cocamide DEA, soyamide DEA, lauramide DEA, lauramide MEA, lauramide MIPA, oleamide MIPA, ricinoleamide DEA, stearamide MEA, stearamide DEA, isostearamide MEA, isostearamide DEA, myristamide MEA, lauramide MEA, capramide DEA, stearamide DEA, oleylamide DEA, myristamide DEA, or tallowamide DEA, or combinations thereof.

According to various aspects of the present disclosure, surfactants may also be cationic surfactants present in an amount preferably ranging from 0 weight percent to about 5 weight percent and more preferably from 0 weight percent to about 1 weight percent, based on the total weight of the composition. In some instances, examples of cationic surfactants include quaternary ammonium compounds.

A more extensive list of surfactants is described for example in U.S. Pat. No. 5,955,066, and Mccutcheon's Emulsifiers & Detergents, North American Edition and International Edition, 1999 Annuals, published by Mccutcheon's Division, MC Publishing Company, Glen Rock, N.J. (1999). The complete disclosure of these documents are incorporated herein by reference in their entireties.

Hand soap compositions according to the disclosure comprise water as a carrier. In some instances, water is present in an amount of at least about 40 weight percent, preferably from about 50 weight percent to about 90 weight percent, and more preferably from about 60 weight percent to about 80 weight percent, based on the total weight of the hand soap composition.

In addition to water, hand soap compositions according to the disclosure may also contain other carriers such as organic solvents. If used as a carrier, such organic solvents, are present in such hand soap composition in an amount of no more than about 5 weight percent and more preferably in an amount of from about 0.1 to about 1 weight percent based on the total weight of the composition. In some instances, non-limiting examples of organic solvents include C₂ to C₆ monoalcohols, such as ethanol, propanol, isopropanol, or tert-butyl alcohol; ethylene glycol; ethylene glycol monomethyl ether; ethylene glycol monoethyl ether; ethylene glycol monobutyl ether; ethylene glycol monoethyl ether acetate; polyethylene glycol, propylene glycol; propylene glycol monomethyl ether; dipropylene glycol monomethyl ether; glycerol; diethylene glycol; glycerin; or any combination thereof. In some instances, the hand soap compositions according to the disclosure are formulated as monoalcohol-free compositions.

In some instances, hand soap compositions according to the disclosure comprise a primary humectant as a moisturizing component. In some instances, the primary humectant does not comprise any reducing sugar. In some instances, illustrative examples of primary humectants include, but are not limited to, glycerin, propylene glycol, butylene glycol, hexylene glycol, glyceryl triacetate, and mixtures thereof. In some instances, the primary humectant is present in the hand soap compositions according to the disclosure in an amount of no more than about 5 weight percent and more preferably in an amount of from about 0.1 to about 1.5 weight percent, based on the total weight of the soap composition.

In some instances, hand soap compositions according to the disclosure further comprise an amine source and a sustainable raw material including a reducing sugar. In some instances, suitable amine sources can be any compound that comprises at least one amine functional group. In some instances, for example, the amine source can comprise, but is not limited to, a protein, an amine containing surfactant (e.g., for example, a quaternary ammonium compound, an ethanolamine compound (monoethanolamine, diethanolamine, triethanolamine or mixtures thereof), cetrimonium chloride, dimethyl myristamine and lauramidopropylamine oxide), a chelating agent (such as, for example ethylenediamine, ethylenediaminetetraacetic (EDTA), an amine containing enzyme, a hydrolyzed protein (such as, for example, hydrolyzed keratin or hydrolyzed wheat proteins), or any combination thereof. In some instances, the amine source is present in hand soap compositions in an amount from about 0.05 weight percent to about 30 weight percent, with an amount from about 0.25 weight percent to about 20 weight percent being more preferable, and an amount from about 0.5 weight percent to about 15 weight percent being most preferable. In some instances, in which the amine source is an amine containing surfactant, the amine containing surfactant is present in hand soap compositions in an amount from about 1 weight percent to about 20 weight percent. In some instances in which the amine source is other than an amine containing surfactant, the non-surfactant amine source is present in hand soap compositions in an amount from about 0.05 weight percent to about 5 weight percent.

Ethanolamine compounds contain a group of amino alcohols. Ethanolamine compounds can be used as a surfactant in some hand soap compositions according to the disclosure. Ethanolamine compounds can aid in the removal of dirt, grease and stains. Illustrative examples of ethanolamine compounds include, but are not limited to, monoethanolamine, diethanolamine, triethanolamine or mixtures thereof. In some instances, the ethanolamine compound is present in an amount ranging from about 0.5 to about 10 wt % of hand soap compositions according to the disclosure. In some instances, the ethanolamine is present in an amount ranging from about 1.0 to about 5.0 wt % of hand soap compositions according to the disclosure.

A chelating agent is a compound containing a ligand (typically organic) that can react with metal ions to form a stable, water-soluble complex. Chelating agents can also be referred to as chelants, chelators or sequestering agents. Chelating agents can have a ring-like center which forms at least two bonds with the metal ion. In some instances, the chelate is any compound that includes at least an amine functional and that can be used in hand soap formulations. In some instances, a chelating agent is present in an amount ranging from about 0.1 to about 5 wt % of hand soap compositions according to the disclosure. In some instances, the chelating agent is present in an amount ranging from about 0.1 to about 2.5 wt % of hand soap compositions according to the disclosure. Exemplary chelates that can be employed as an amine source include, but are not limited to, tetrasodium iminodisuccinate, ethylenediamine, ethylenediaminetetraacetic (EDTA) or mixtures thereof. In some instances, the chelating agent is N,N-bis(carboxymethyl)-L-glutamic acid tetrasodium salt. Chelating agents are sometimes used as water softeners in hand soap compositions. In some instances, the chelating agent is iminodisuccinate (IDS), ethylenediaminetetraacetic acid (EDTA), nitrilotriacetic acid, diethylenetriaminepenta (methylenephosphonic acid), nitrilotris (methylenephosphonic acid), ethylenediamine-N,N′-disuccinic acid (EDDS), hydroxyethylenediaminetriacetic acid (HEDTA), N,N-bis(carboxymethyl)-L-glutamic acid tetrasodium salt, Alanine, N,N-bis(carboxymethyl)-alanine, or other chelating compounds comprising an amine group.

Amino acids are organic compounds that contain both amino and carboxylic functional groups. Exemplary amino acids that can be used as the amine source include, but are not limited to, arginine, histidine, lysine, aspartic acid, glutamic acid, serine, threonine, asparagine, glutamine, cysteine, selenocysteine, glycine, proline, alanine, valine, isoleucine, methionine, phenylalanine, tyrosine, tryptophan or any combination thereof.

In some instances, amine-containing enzymes (i.e., enzymes that containing at least one amine functionality) can also be used as the amine source.

As mentioned above and in some instances, a sustainable raw material comprising a reducing sugar can be present in hand soap compositions according to the disclosure. In some instances, these reducing sugars can be a residual contaminant present in the sustainable raw material, or it can be a moiety of the sustainable raw material that can be released upon degradation. In some instances, for example, a reducing sugar can be contained in a sustainable solvent, a sustainable polymer and/or a secondary humectant that can be used in formulating hand soap compositions according to the disclosure. In some instances, the hand soap compositions can further comprise a sustainable polymer that includes a reducing sugar monomer. In some instances, the sustainable polymer can be present in hand soap compositions according to the disclosure in an amount ranging from about 0.01 weight percent to about 10 weight percent. In some instances, the sustainable polymer can be present in hand soap compositions in an amount ranging from about 0.25 weight percent to about 2 weight percent. In some instances, the sustainable polymer can be present in hand soap compositions in an amount ranging from about 0.5 weight percent to about 1 weight percent. In some instances, the polymer including the reducing sugar can further comprise a polysaccharide.

A polysaccharide is a polymer made of chains of monosaccharides that are joined by glycosidic linkages. In some instances, a polysaccharide preferably comprises more than ten monosaccharide units. In some instances, polysaccharides may be linear or branched. In some instances, linear polysaccharides are preferably rigid polymers, while branched polysaccharides are typically soluble in water. In some instances, a polysaccharide includes starches, glycogens, galactogens, cellulose and/or chitin. In some instances, an illustrative example of a polysaccharide that can be used in the present disclosure as the sustainable polymer includes, but is not limited to, poly alpha-1,6-glucan.

In some instances, the sustainable polymer comprises a reducing sugar monomer. In some instances, the reducing sugar monomer can be a contaminate (residual) or it can be a moiety of the sustainable polymer that can be released upon degradation. In some instances, in hand soap compositions comprising an amine source, the reducing sugar monomer (residual or released) and the amine source can react together under heat to cause unwanted yellowing of the hand soap composition. In some instances, the reducing sugar monomer is present in the sustainable polymer in an amount of from about 0.0001 weight percent to about 1 weight percent, with an amount of from about 0.005 weight percent to about 0.5 weight percent being more preferable. A reducing sugar monomer is any sugar monomer that can act as a reducing agent. In some instances, the reducing sugar monomer can comprise a monosaccharide or disaccharide. In some instances, exemplary reducing sugar monomers that can be contained in the polymer include, but are not limited to, fructose, glucose, hexose, lactose, glyceraldehyde, arabinose, maltose, galactose, ribose, xylose, cellobiose or combinations thereof. In some instances, the reducing sugar monomer can be present in the raw materials that are used in forming the sustainable polymer mentioned above.

In some instances, hand soap compositions according to the disclosure can comprise at least one sustainable solvent that comprises a reducing sugar (residual or moiety). In some instances, the sustainable solvent is present in hand soap composition sin an amount of from about 0.01 weight percent to about 60 weight percent, with an amount of from about 1 weight percent to about 30 weight percent being more preferable, and an amount of from about 5 weight percent to about 20 weight percent being most preferable. In some instances, illustrative sustainable solvents including a reducing sugar that can be employed in hand soap compositions according to the disclosure comprise, but are not limited to, sorbitol, xylitol, mannitol, lactitol, isomalt, maltitol, a hydrogenated starch hydrolysate, sucrose, stachyose, verbascose, trehalose, raffinose, or any combination thereof. In some instances, the reducing sugar is present in the sustainable solvent in an amount of from about 0.0001 weight percent to about 1 weight percent, with an amount of from about 0.005 weight percent to about 0.5 weight percent being more preferable. In some instances, the reducing sugar can be present in the raw materials that are used in forming the at least one sustainable solvent mentioned above.

In some instances, hand soap compositions according to the disclosure can further comprise a secondary humectant that comprises a reducing sugar (residual or moiety). In some instances, the secondary humectant is present in hand soap compositions in an amount of from about 0.01 weight percent to about 5 weight percent, with an amount of from about 0.1 weight percent to about 2 weight percent being more preferable, and an amount of from about 0.5 weight percent to about 1 weight percent being even more preferable. In some instances, an illustrative secondary humectant including a reducing sugar that can be employed in hand soap compositions according to the disclosure include, but are not limited to, alkyl glucose ethers (in some instances, C₁₋₁₀ alkyl glucose ethers are preferred). In one example, the alkyl glycol ether is methyl glucose ether. In some instances, the reducing sugar is present in the secondary humectant in an amount ranging from about 0.0001 weight percent to about 1 weight percent, with an amount of from about 0.005 weight percent to about 0.5 weight percent being more preferable based on the weight of the composition.

In some instances, hand soap compositions according to the disclosure can further comprise any combination of a sustainable solvent that includes a reducing sugar (residual or moiety), a sustainable polymer that includes a reducing sugar monomer (residual or moiety), and a secondary humectant that contains a reducing sugar.

In some instances, hand soap compositions according to the disclosure comprise a primary amine source comprising a fermented raw material that includes a reducing sugar. In some instances, examples of such primary amine sources include, but are not limited to, biosurfactants (such as, for example, sophorolipid or rhamnolipid). In some instances, the reducing sugar can be a contaminate (residual) or it can be a moiety of the primary amine source that can be released upon degradation. In some instances, in hand soap compositions including such a primary amine source, the reducing sugar (residual or released) and the amine of the primary amine source can react together under heat to cause unwanted yellowing of the hand soap composition.

In some instances, the reducing sugar is present in the primary amine source in an amount of from about 0.0001 weight percent to about 1 weight percent, with an amount of from about 0.005 weight percent to about 0.5 weight percent being more preferable. A reducing sugar is any sugar that can act as a reducing agent. In some instances, the reducing sugar comprises a monosaccharide or disaccharide. Exemplary reducing sugars that can be contained in the amine containing surfactant include, but are not limited to, fructose, glucose, hexose, lactose, glyceraldehyde, arabinose, maltose, galactose, ribose, xylose, cellobiose, or any combination thereof. In some instances, the primary amine source is present in the hand soap composition in an amount from about 5 weight percent to about 30 weight percent, with an amount from about 10 weight percent to about 25 weight percent being more preferable, and an amount from about 12 weight percent to about 20 weight percent being most preferable.

In some instances, hand soap compositions according to the disclosure can comprise the primary amine source, a secondary amine source and the sustainable raw material including the reducing sugar as mentioned above. In some instances, the secondary amine source is an amine source in which non-fermented raw materials (which do include a reducing sugar) are used in forming the same. In some instances, the secondary amine source includes any compound that comprises at least one amine functional group and that can be used in hand soap compositions. For example, the secondary amine source can include, but is not limited to, a protein, an amine containing surfactant (e.g., for example, a quaternary ammonium compound, an ethanolamine compound (monoethanolamine, diethanolamine, triethanolamine or mixtures thereof), cetrimonium chloride, dimethyl myristamine and lauramidopropylamine oxide), a chelating agent (such as, for example ethylenediamine, ethylenediaminetetraacetic (EDTA), an amine containing enzyme, a hydrolyzed protein (such as, for example, hydrolyzed keratin or hydrolyzed wheat proteins) or any combination thereof. In some instances, the secondary amine source is present in hand soap compositions according to the disclosure in an amount from about 0.05 weight percent to about 30 weight percent, with an amount from about 0.25 weight percent to about 20 weight percent being more preferable, and an amount from about 0.5 weight percent to about 15 weight percent being even more preferable.

Hand soap compositions according to the disclosure comprise a Maillard reaction inhibiting enzyme. In some instances, the Maillard reaction inhibiting enzyme is any enzyme that can prevent yellowing of the hand soap composition by inhibiting the reaction (i.e., Maillard reaction) between the reducing sugar and the amine (from the amine source, primary amine source and/or secondary amine source mentioned above). In some instances, the Maillard reaction inhibiting enzyme is present in hand soap compositions according to the disclosure in an amount of from about 0.001 weight percent to about 3 weight percent, with an amount of from about 0.005 weight percent to about 0.5 weight percent being more preferable, and an amount from about 0.01 weight percent to about 0.1 weight percent being even more preferable. In some instances, the Maillard reaction inhibiting enzyme is a reducing sugar oxidation enzyme. Such oxidation enzymes oxidize the reducing sugar that is present and prevent subsequent yellowing of hand soap compositions from occurring. In some instances, the Maillard reaction inhibiting enzyme is an enzyme that reacts with fructose and glucose. In some instances, illustrative examples of Maillard reaction inhibiting enzymes that can prevent yellowing of hand soap compositions of the disclosure include, but are not limited to, fructosamine oxidase, fructosamine kinase, hexose oxidase, or carbohydrate oxidase. It is noted that within the range mentioned above, the Maillard reaction inhibiting enzyme can prevent the Maillard reaction from occurring throughout the entire shelf-life of the hand soap composition.

The Maillard reaction inhibiting enzyme can be added to a raw material composition that contains at least one of the sustainable raw materials mentioned above utilizing techniques that are well known to those skilled in the art. In some instances, the addition includes a metered addition. The addition can be performed with continuous stirring. In some instances, the addition is typically performed or around room temperature, e.g., from about 20° C. to about 30° C. This addition provides the formulations in accordance with various aspects of the disclosure. In addition to the Maillard reaction inhibiting enzyme and the raw material composition mentioned above, formulations according to the disclosure can include additional components as described below. The additional components can be present in a raw material composition that includes a sustainable raw material and a reducing sugar, or they can be added after forming the raw material composition.

Hand soap compositions according to the disclosure are formulated to have a pH that is dermatologically compatible with skin. In some instances, hand soap compositions according to the disclosure are formulated to have a pH ranging from about 2 to about 10. In some instances, hand soap compositions according to the disclosure are formulated to have a pH ranging from about 2.5 to about 9, preferably from about 3.0 to about 8, more preferably from about to 3.5 to about 7.5, more preferably from about 4.0 to about 6.5, and most preferably about 5.0 to 5.5. If necessary, the pH of hand soap compositions according to the disclosure can be adjusted with any of a number of commonly used cosmetic ingredients. Non-limiting examples of suitable pH adjusters include acids for decreasing the pH including inorganic acids such as phosphoric acid, nitric acid, or hydrochloric acid, or organic acids such as citric acid, lactic acid or tartaric acid; or basic compounds for increasing the pH, including organic amines such as triethanolamine, ethanolamine, or aminomethylpropanol, alkali metal or alkaline earth metal hydroxides such as sodium hydroxide or potassium hydroxide; ammonium hydroxide, basic amino acids such as arginine, sodium hydroxymethylglycinate, or combinations thereof.

In some instances, hand soap compositions according to the disclosure can further comprise an antibacterial active material. In some instances, the antibacterial active material that can be employed in the present disclosure includes, but is not limited to, triclosan, bisguanidine, diphenyl compounds, benzyl alcohols, trihalocarbanilides, quaternary ammonium compounds, ethoxylated phenols, phenolic compounds, halo-substituted phenolic compounds, p-chloro-m-xylenol, include phenolic compounds, 2-hydroxydiphenyl compounds, chlorophenols (o-, m-, p-), 2,4-dichlorophenol, p-nitrophenol, picric acid, xylenol, p-chloro-m-xylenol, cresols (o-, m-, p-), p-chloro-m-cresol, pyrocatechol, resorcinol, 4-n-hexylresorcinol, pyrogallol, phloroglucin, carvacrol, thymol, p-chlorothymol, o-phenylphenol, o-benzylphenol, p-chloro-o-benzylphenol, phenol, 4-ethylphenol, 4-phenolsulfonic acid, diphenyl compounds, hexachlorophene, tetrachlorophene, dichlorophen, 2,3-dihydroxy-5,5′-dichlorodiphenyl sulfide, 2,2′-dihydroxy-3,3′,5,5′-tetrachlorodiphenyl sulfide, 2,2′-dihydroxy-3,5′,5,5′,6,6′-hexachlorodiphenyl sulfide, and 3,3′-dibromo-5,5′-dichloro-2,2′-dihydroxydiphenylamine. In some instances, the antibacterial active material can be present in an amount of from 0 weight percent to about 2 weight percent, based on the total weight of the hand soap composition.

In some instances, inorganic electrolyte salts may also be added to hand soap compositions according to the disclosure in an amount of from 0 weight percent to about 2 weight percent, based on the total weight. In some instances, electrolyte salts may be added, for example, to thicken the composition. In some instances, inorganic electrolyte salts that can be used are any water-soluble alkali metal, ammonium or alkaline earth metal salt, for example the fluorides, chlorides, bromides, sulfates, phosphates, nitrates or hydrogen carbonates, providing that they are soluble in water at 20° C. in a quantity of at least 1 percent by weight. In some instances, inorganic electrolyte salts such as sodium chloride and magnesium chloride are especially preferred.

In some instances, hand soap compositions according to the disclosure further comprise optional additive components, one or more sequestrants (i.e., chelants) such as ethylene diamine tetraacetic acid (EDTA), methylgylcinediactetic acid (MGDA), or phosphonic acids; fragrances; other nonactive ingredients such as panthenol, allantoin, pyrrolidone carboxylic acids or salts thereof; plant extracts; vitamins such as Vitamins A, B, C, or E, or combinations thereof; preservatives such methylchloroisothiazolinone, methylisothiazolinone, paraben, or diazolidinyl urea or combinations thereof; light stabilizers; dyes (or colorants); pearlescers (or opacifies), such as ethylene glycol monostearate (EGMS) or distearate; antioxidants; viscosifers (or viscosity builder) such as, for example, alkanol amine, salt, amine oxide; foam boasters including, for example, amino oxide, sacosinate, or lactylate; foam stabilizers such as, for example, lactylate or gum; active additives such as, for example, zinc pyrithione or salicylic acid; thickeners such as polysaccharides, including for example, xanthan gum, guar gum, agar gum, alginates, tyloses, or celluloses such as carboxymethyl cellulose, or hydroxyethyl cellulose; polyethylene glycol monoesters or diesters of fatty acids having a molecular weight of at least about 200,000; poly(meth)acrylic acid or salts thereof; polyvinyl alcohol; or fatty alcohol ethoxylates; or any combination of any of the foregoing additives. In some instances, the selection of these optional additives will depend upon the hand soap composition that is desired to be formulated. In some instances, the optional additives are present individually in the hand soap composition in an amount of no more than about 2 weight percent and more preferably in an amount of no more than about 0.5 weight percent based on the total weight of the composition.

Hand soap compositions according to the disclosure can be used for cleaning hands using various cleaning methodologies. In some instances, hand soap compositions according to the disclosure are preferably applied to skin that has first been wetted with water. Following application of the hand soap composition to the skin, the composition is rinsed from the skin with water to remove any undesirable substances such as dirt or sebum. The hand soap composition has the advantage of cleaning the skin.

The hand soap compositions described herein can be formulated utilizing techniques well known to those skilled in the art. In some instances, the various components that provide a hand soap composition according to the disclosure can be added in any order. For example, an amine source can be added to a reactor vessel including at least one surfactant, a humectant, water and a sustainable raw material including a reducing sugar, and thereafter a Maillard reaction inhibiting enzyme can be added to the vessel. In some instances, the addition is a metered addition. In some instances, the addition of the various components that provide a hand soap composition according to the disclosure can be performed with continuous stirring. In some instances, the addition is performed at room temperature, e.g., from about 20° C. to about 30° C. In some instances, an enzyme enhancing agent can be added during the formulation process to improve the performance of the Maillard reaction inhibiting enzyme. In some instances, the Maillard reaction inhibiting enzyme can be added to a previously formulated hand soap composition.

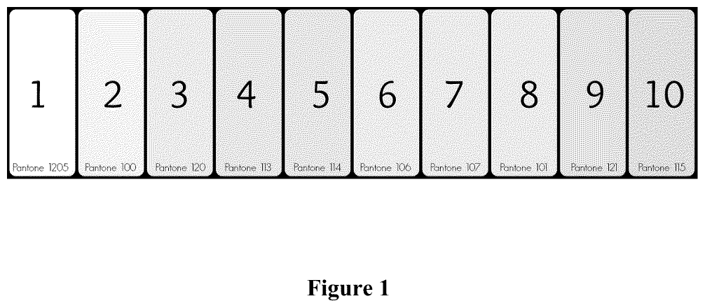

In accordance with various aspects of the disclosure, the yellowing in a hand soap composition, such as a hand soap composition according to various aspects of the disclosure, can be measured through a pantone color system. FIG. 1 displays the numerical representation of each color of yellow. Table 1 is also the tabular representation of the FIG. 1.

TABLE 1

| Numerical Representation | Pantone Number |
|---|---|
| 1 | Pantone 1205 |
| 2 | Pantone 100 |
| 3 | Pantone 120 |
| 4 | Pantone 113 |
| 5 | Pantone 114 |
| 6 | Pantone 106 |
| 7 | Pantone 107 |
| 8 | Pantone 101 |
| 9 | Pantone 121 |
| 10 | Pantone 115 |

### EXAMPLES

Examples have been set forth below for the purpose of further illustrating the present disclosure. The scope of the present disclosure is not limited to any of the examples set forth herein.

### Prophetic Example 1

Hand soap compositions can be prepared by mixing the components in the amounts listed in Table 2. Notably, the hand soap compositions can be batched using a standard overhead mixer, target pH 4.0 to 5.5 and a target viscosity between 3000-6000 cps. Formula 1 and Formula 2 of Table 2 are substantially identical in composition except that Formula 2 includes a Maillard reaction inhibiting enzyme as described herein, while the Maillard reaction inhibiting enzyme is omitted from Formula 1. In Formula 2 below, the Maillard reaction inhibiting enzyme can be added prior to reducing the pH to the target value mentioned above.

TABLE 2

| | Wt % of Formula 1 | Wt % of Formula 2 |
|---|---|---|
| Component | (CE1) | (Inventive) |
| Water, demineralized | q/s to 100 | q/s to 100 |
| Benzalkonium | 0.1 to 1 | 0.1 to 1 |
| chloride (anti- | | |
| bacterial agent) | | |
| Lauramine oxide | 1 to 10 | 1 to 10 |
| (primary surfactant) | | |
| Lauryl/myristyl | 1 to 10 | 1 to 10 |
| amidoamine (primary | | |
| surfactant) | | |
| Cetyl trimethyl | 1 to 10 | 1 to 10 |
| ammonium chloride | | |
| (secondary surfactant) | | |
| Methyl glucose ether | 0.1 to 1 | 0.1 to 1 |
| (thickening polymer) | | |
| Maillard reaction | 0 | 0.001 to 1 |
| inhibiting enzyme | | |
| pH adjuster | As needed to | As needed to |
| (ex. Citric acid) | pH 4 to 5.5 | pH 4 to 5.5 |
| Preservative | 0.1 to 1 | 0.1 to 1 |
| (ex. Sodium | | |
| benzoate) | | |
| Glycerin (primary | 1 to 5 | 1 to 5 |
| humectant) | | |
| Thickener | 1 to 5 | 1 to 5 |
| (ex. NaCl) | | |
| Additives | 0.1 to 1 | 0.1 to 1 |

### Prophetic Example 2

Hand soap compositions can be prepared by mixing the components in the amounts listed in Table 3. Notably, the hand soap compositions can be batched using a standard overhead mixer, target pH 4.0 to 5.5 and a target viscosity between 3000-6000 cps. Formula 3 and Formula 4 of Table 3 are substantially identical in composition except that Formula 4 includes a Maillard reaction inhibiting enzyme as described herein, while the Maillard reaction inhibiting enzyme is omitted from Formula 3.

TABLE 3

| | Wt % of Formula 3 | Wt % of Formula 4 |
|---|---|---|
| Component | (Comparative) | (Inventive) |
| Water, demineralized | q/s to 100 | q/s to 100 |
| Benzalkonium chloride (anti- | 0.1 to 1 | 0.1 to 1 |
| bacterial agent) | | |
| Lauramine oxide (primary | 1 to 10 | 1 to 10 |
| surfactant) | | |
| Lauryl/myristyl amidoamine | 1 to 10 | 1 to 10 |
| (primary surfactant) | | |
| Cetyl trimethyl ammonium | 1 to 10 | 1 to 10 |
| chloride (secondary | | |
| surfactant) | | |
| Methyl glucose ether | 0.1 to 1 | 0.1 to 1 |
| (thickening polymer) | | |
| Biosurfactant including | 0.1 to 5 | 0.1 to 5 |
| reducing sugar | | |
| Maillard reaction inhibiting | 0 | 0.001 to 1 |
| enzyme | | |
| pH adjuster | As needed to | As needed to |
| (ex. citric acid) | pH 4 to 5.5 | pH 4 to 5.5 |
| Preservative | 0.1 to 1 | 0.1 to 1 |
| (ex. Sodium benzoate) | | |
| Glycerin (primary | 1 to 5 | 1 to 5 |
| humectant) | | |
| Thickener | 1 to 5 | 1 to 5 |
| (ex. NaCl) | | |
| Additives | 0.1 to 1 | 0.1 to 1 |

### Prophetic Example 3

Hand soap compositions can be prepared by mixing the components in the amounts listed in Table 4. Notably, the hand soap compositions can be batched using a standard overhead mixer, target pH 2 to 12. Formula 5 and Formula 6 of Table 4 are substantially identical in composition except that Formula 6 includes a Maillard reaction inhibiting enzyme as described herein, while the Maillard reaction inhibiting enzyme is omitted from Formula 5.

TABLE 4

| | Wt % of Formula 5 | Wt % of Formula 6 |
|---|---|---|
| Component | (Comparative) | (Inventive) |
| Water, demineralized | q/s to 100 | q/s to 100 |
| Benzalkonium chloride | 0.1 to 1 | 0.1 to 1 |
| (anti-bacterial agent) | | |
| Lauramine oxide (primary | 1 to 5 | 1 to 5 |
| surfactant) | | |
| Cocamidopropyl betaine | 1 to 5 | 1 to 5 |
| (primary surfactant) | | |
| Cetyl trimethyl | 1 to 5 | 1 to 5 |
| ammonium chloride | | |
| (secondary surfactant) | | |
| Biosurfactant including | 0.1 to 5 | 0.1 to 5 |
| reducing sugar | | |
| Maillard reaction | 0 | 0.001 to 1 |
| inhibiting enzyme | | |
| pH adjuster | As needed to | As needed to |
| (ex. citric acid) | pH 4 to 5.5 | pH 4 to 5.5 |
| Preservative | 0.1 to 1 | 0.1 to 1 |
| (ex. Sodium benzoate) | | |
| Glycerin (primary | 1 to 5 | 1 to 5 |
| humectant) | | |
| Additives | 0.1 to 1 | 0.1 to 1 |

### Prophetic Example 4

Hand soap compositions can be prepared by mixing the components in the amounts listed in Table 5. Notably, the hand soap compositions can be batched using a standard overhead mixer, target pH 2 to 12. Formula 7 and Formula 8 of Table 5 are substantially identical in composition except that Formula 8 includes a Maillard reaction inhibiting enzyme as described herein, while the Maillard reaction inhibiting enzyme is omitted from Formula 7.

TABLE 5

| | Wt % of Formula 7 | Wt % of Formula 8 |
|---|---|---|
| Component | (Comparative) | (Inventive) |
| Water, demineralized | q/s to 100 | q/s to 100 |
| Benzalkonium chloride | 0.1 to 1 | 0.1 to 1 |
| (anti-bacterial agent) | | |
| Lauramine oxide (primary | 1 to 5 | 1 to 5 |
| surfactant) | | |
| Cocamidopropyl betaine | 1 to 5 | 1 to 5 |
| (primary surfactant) | | |
| Cetyl trimethyl ammonium | 1 to 5 | 1 to 5 |
| chloride (secondary | | |
| surfactant) | | |
| Biopolymer containing | 0.1 to 5 | 0.1 to 5 |
| glucose polymer and/or | | |
| residual reducing sugars | | |
| Maillard reaction inhibiting | 0 | 0.001 to 1 |
| enzyme | | |
| pH adjuster | As needed to | As needed to |
| (ex. Citric acid) | pH 4 to 5.5 | pH 4 to 5.5 |
| Preservative | 0.1 to 1 | 0.1 to 1 |
| (ex. Sodium benzoate) | | |
| Glycerin (primary | 1 to 5 | 1 to 5 |
| humectant) | | |
| Additives | 0.1 to 1 | 0.1 to 1 |

### Example 5

Formulas 9 and 10 were created by mixing the above components together using a standard overhead mixer at room temperature for 1 hour. Glucose was added to illustrate the effects of a reducing sugar. The formulations of formulas 9 and 10 are seen in Table 6.

TABLE 6

| | | Formula 9 | Formula 10 |
|---|---|---|---|
| | | (Comparative) | (Inventive) |
| Ingredient | Active % | Wt % | Wt % |
| Water | 100 | 72.62 | 72.52 |
| Glucose | 100 | 0.50 | 0.50 |
| Hexose oxidase | 100 | 0.00 | 0.10 |
| Benzalkonium | 50 | 0.26 | 0.26 |
| Chloride | | | |
| Amine Oxides, Cetyl | 30 | 13.70 | 13.70 |
| Trimethyl Ammonium | | | |
| Chloride | | | |
| PEG-120 Methyl | 100 | 0.75 | 0.75 |
| Glucose Trioleate, | | | |
| Propanediol | | | |
| Glycerin, Na | N/A | 7.17 | 7.17 |
| Benzoate, Sodium | | | |
| Chloride, Trisodium | | | |
| Ehtylenediamine | | | |
| Disuccinate, | | | |
| Monoethanolamine | | | |
| Citric Acid (pH adjust | 50 | 5.00 | 5.00 |
| to 5.5, then QS) | | | |
| Total | | 100.00 | 100.00 |

Formulas 9 and 10 were tested for yellowing with the results displayed below in Table 7. Formula 10, with the addition of the Maillard reaction inhibiting enzyme, prevented yellowing in the hand soap composition.

| Formula # (Storage Conditions) | Pantone Color ranking (0-10)* |
|---|---|
| Formula 9 (1-week at 24° C.) | 2 |
| Formula 10 (1-week at 24° C.) | 0 |
| Formula 9 (1-week at 52° C.) | 4 |
| Formula 10 (1-week at 52° C.) | 0 |

### Example 6

Formulas 11 and 12 were created by mixing the above components together using a standard overhead mixer at room temperature for 1 hour. Glucose was added to illustrate the effects of a reducing sugar. The formulations of formulas 11 and 12 are seen in Table 8.

TABLE 8

| | | Formula 11 | Formula 12 |
|---|---|---|---|
| | | (Comparative) | (Inventive) |
| Ingredient | Active % | Wt % | Wt % |
| Water | 100 | 83.12 | 83.02 |
| Glucose | 100 | 0.50 | 0.50 |
| Hexose oxidase | 100 | 0.00 | 0.10 |
| Benzalkonium | 50 | 0.26 | 0.26 |
| Chloride | | | |
| Glycerin, Na | 100 | 4.77 | 4.77 |
| Benzoate, Trisodium | | | |
| Ehtylenediamine | | | |
| Disuccinate, | | | |
| Monoethanolamine | | | |
| Amine oxide, | ~30 to 40 | 6.35 | 6.35 |
| Cocamindopropyl | | | |
| betaine, Cetyl | | | |
| Trimethyl Ammonium | | | |
| Chloride | | | |
| Citric Acid (pH adjust | 50 | 5.00 | 5.00 |
| to 5.5, then QS) | | | |
| Total | | 100.00 | 100.00 |

Formulas 11 and 12 were tested for yellowing with the results displayed below in Table 9. Formula 12, with the addition of the Maillard reaction inhibiting enzyme, prevented yellowing in the hand soap composition.

TABLE 9

| Formula # (Storage Conditions) | Pantone Color ranking (0-10)* |
|---|---|
| Formula 11 (1-week at 24° C.) | 1 |
| Formula 12 (1-week at 24° C.) | 0 |
| Formula 11 (1-week at 52° C.) | 8 |
| Formula 12 (1-week at 52° C.) | 4 |

While the present disclosure has been particularly shown and described with respect to preferred embodiments thereof, it will be understood by those skilled in the art that the foregoing and other changes in forms and details may be made without departing from the spirit and scope of the present disclosure. It is therefore intended that the present disclosure not be limited to the exact forms and details described and illustrated, but fall within the scope of the appended claims.

1. A hand soap composition comprising:
at least one surfactant;
an amine source;
a sustainable raw material including a reducing sugar;
a Maillard reaction inhibiting enzyme that inhibits reaction between the amine source and the reducing sugar;
a primary humectant; and
at least 40 weight percent water, based on the total weight of the hand soap composition.

2. The hand soap composition of claim 1, wherein the at least one surfactant comprises a primary surfactant, where the primary surfactant is present in an amount ranging from about 1 weight percent to about 40 weight percent, based on the total weight of the hand soap composition.

3. The hand soap composition of claim 2, wherein the at least one surfactant further comprises a secondary surfactant, wherein the secondary surfactant is present in an amount ranging from about 0.1 weight percent to about 15 weight percent, based on the total weight of the hand soap composition.

4. The hand soap composition of claim 1, wherein the amine source comprises a protein, an amine containing surfactant, a chelating agent, an amine containing enzyme, or a hydrolyzed protein, or any combination thereof.

5. The hand soap composition of claim 1, wherein the amine source is present in an amount ranging from about 0.05 weight percent to about 30 weight percent, based on the total weight of the hand soap composition.

6. The hand soap composition of claim 1, wherein the sustainable raw material comprises a sustainable polymer, a sustainable solvent, or a secondary humectant, or any combination thereof.

7. The hand soap composition of claim 6, wherein the sustainable solvent comprises sorbitol, xylitol, mannitol, lactitol, isomalt, maltitol, a hydrogenated starch hydrolysate, sucrose, stachyose, verbascose, trehalose, or raffinose, or any combination thereof.

8. The hand soap composition of claim 6, wherein the sustainable polymer comprises a starch, a glycogen, a galactogen, a cellulose, or a chitin, or any combination thereof.

9. The hand soap composition of claim 1, wherein the Maillard reaction inhibiting enzyme comprises fructosamine oxidase, fructosamine kinase, or carbohydrate oxidase, or any combination thereof.

10. The hand soap composition of claim 1, wherein the Maillard reaction inhibiting enzyme is present in an amount ranging from about 0.001 weight percent to about 3 weight percent, based on the total weight of the hand soap composition.

11. A hand soap composition comprising:
at least one surfactant;
a primary amine source comprising a fermented raw material, where the fermented raw material includes a reducing sugar;
a Maillard reaction inhibiting enzyme that inhibits reaction between the primary amine source and the reducing sugar;
a primary humectant; and
at least 40 weight percent water, based on the total weight of the hand soap composition.

12. The hand soap composition of claim 11, wherein the at least one surfactant comprises a primary surfactant, where the primary surfactant is present in an amount from about 1 weight percent to about 40 weight percent, based on the total weight of the hand soap composition.

13. The hand soap composition of claim 12, wherein the at least one surfactant further comprises a secondary surfactant, where the secondary surfactant is present in an amount ranging from about 0.1 weight percent to about 15 weight percent, based on the total weight of the hand soap composition.

14. The hand soap composition of claim 11, wherein the primary amine source comprises a biosurfactant, an alkyl glucose ether, or a combination of a biosurfactant and an alkyl glucose ether.

15. The hand soap composition of claim 11, wherein the primary amine source is present in an amount from about 0.05 weight percent to about 30 weight percent, based on the total weight of the hand soap composition.

16. The hand soap composition of claim 11, wherein the Maillard reaction inhibiting enzyme comprises fructosamine oxidase, fructosamine kinase, or carbohydrate oxidase, or any combination thereof.

17. The hand soap composition of claim 11, wherein the Maillard reaction inhibiting enzyme is present in an amount ranging from about 0.001 weight percent to about 3 weight percent, based on the total weight of the hand soap composition.

18. The hand soap composition of claim 11, further comprising at least one of a sustainable solvent including a reducing sugar, a sustainable polymer including a reducing sugar, and a secondary humectant including a reducing sugar.

19. The hand soap composition of claim 18, wherein the sustainable solvent comprises sorbitol, xylitol, mannitol, lactitol, isomalt, maltitol, a hydrogenated starch hydrolysate, sucrose, stachyose, verbascose, trehalose, or raffinose, or any combination thereof.

20. The hand soap composition of claim 18, the sustainable polymer comprises a starch, a glycogen, a galactogen, a cellulose, or a chitin, or any combination thereof.
